(19) United States
(12) Patent Application Publication
(10) Pub. No.: US 2024/0335124 A1
(43) Pub. Date: Oct. 10, 2024

(54) PHYSIOLOGICAL INFORMATION MEASUREMENT DEVICE AND PHYSIOLOGICAL INFORMATION MEASUREMENT METHOD

(71) Applicant: NIHON KOHDEN CORPORATION, Tokyo (JP)

(72) Inventors: Takahiro KURODA, Tokorozawa-shi (JP); Shogo TOTSUKA, Tokorozawa-shi (JP)

(73) Assignee: NIHON KOHDEN CORPORATION, Tokyo (JP)

(21) Appl. No.: 18/615,530

(22) Filed: Mar. 25, 2024

(30) Foreign Application Priority Data
Apr. 7, 2023 (JP) 2023-062853

(51) Int. Cl. A61B 5/022; A61B 5/00; A61B 5/021; A61B 5/0225

(52) U.S. Cl. CPC A61B 5/02225; A61B 5/02141; A61B 5/0225; A61B 5/7221

(57) ABSTRACT

A physiological information measurement device includes: a pressure controller executes a measurement of increasing or decreasing an applied pressure of a cuff with respect to a predetermined site of a subject from an initial value; a pulse detector detects a pulse from a pressure received by the cuff from the predetermined site; a pulse determination unit determines whether the pulse detected by the pulse detector is a correctly detected pulse; a blood pressure calculation possibility determination unit determines whether calculation of a blood pressure of the subject is possible based on the pulse detected by the pulse detector; and a blood pressure calculator calculates the blood pressure of the subject based on a plurality of the pulses determined to be correctly detected by the pulse determination unit.

### CROSS-REFERENCE TO RELATED APPLICATION(S)

This application is based on and claims priority under 35 USC 119 from Japanese Patent Application No. 2023-062853 filed on Apr. 7, 2023, the contents of which are incorporated herein by reference.

### TECHNICAL FIELD

The presently disclosed subject matter relates to a physiological information measurement device, a physiological information measurement method, and a program.

### BACKGROUND ART

There is known a non invasive blood pressure (NIBP) measurement device in which a cuff is wrapped around an upper arm or a lower limb of a subject, and a blood pressure is measured by detecting a pressure vibration generated in the cuff due to a pulse as an oscillation signal while increasing an applied pressure of the cuff to be higher than a systolic blood pressure, and then decreasing the applied pressure. In addition, there is known a non invasive blood pressure measurement device that measures a blood pressure simultaneously with increase of an internal pressure of a cuff for a purpose of shortening a measurement time of the blood pressure (for example, JPH08-322811A).

During the blood pressure measurement, noise may be generated due to a body motion or the like of the subject, and the noise may be erroneously detected as the pulse. In this case, since a locus (envelope) of the obtained oscillation signal changes, it may be difficult to calculate an accurate blood pressure. Therefore, when the blood pressure measurement fails due to the noise, it is necessary to discard the oscillation signal obtained in a blood pressure measurement of a first time and perform a re-measurement. As a result, a burden on the subject is increased, for example, the measurement time is extended.

### SUMMARY

An object of the presently disclosed subject matter is to provide a physiological information measurement device, a physiological information measurement method, and a program capable of reducing a burden on a subject in a blood pressure measurement.

A physiological information measurement device according to a first aspect of the presently disclosed subject matter includes:
a pressure controller configured to execute a measurement of increasing or decreasing an applied pressure of a cuff with respect to a predetermined site of a subject from an initial value;
a pulse detector configured to detect a pulse from a pressure received by the cuff from the predetermined site;
a pulse determination unit configured to determine whether the pulse detected by the pulse detector is a correctly detected pulse;
a blood pressure calculation possibility determination unit configured to determine whether calculation of a blood pressure of the subject is possible based on the pulse detected by the pulse detector; and
a blood pressure calculator configured to calculate the blood pressure of the subject based on a plurality of the pulses determined to be correctly detected by the pulse determination unit,
the pressure controller executes at least the measurement of a second time when the blood pressure calculation possibility determination unit determines that the calculation of the blood pressure is not possible in the measurement of a first time and a re-measurement is necessary, and
the blood pressure calculator calculates the blood pressure of the subject based on a plurality of the pulses determined to be correctly detected by the pulse determination unit in the measurement of a first time or a second and subsequent times.

A physiological information measurement device according to a second aspect of the presently disclosed subject matter includes:
a pressure controller configured to control an applied pressure of a cuff with respect to a predetermined site of a subject;
a pulse detector configured to detect a pulse from a pressure received by the cuff from the predetermined site;
a pulse determination unit configured to determine whether the pulse detected by the pulse detector is a correctly detected pulse; and
a blood pressure calculator configured to calculate a blood pressure of the subject based on a plurality of the pulses determined to be correctly detected by the pulse determination unit, and
the pressure controller controls magnitude of the applied pressure or a duration of application of the applied pressure based on the determination result of the pulse determination unit.

A physiological information measurement method according to a third aspect of the presently disclosed subject matter includes:
executing a measurement of increasing or decreasing an applied pressure of a cuff with respect to a predetermined site of a subject from an initial value;
detecting a pulse from a pressure received by the cuff from the predetermined site;
first determining whether the pulse detected in the detecting is a correctly detected pulse;
second determining whether calculation of a blood pressure of the subject is possible based on the pulse detected in the detecting; and
calculating the blood pressure of the subject based on a plurality of the pulses determined to be correctly detected in the first determining, wherein
in the executing, at least the measurement of a second time is executed when it is determined in the second determining that the calculation of the blood pressure is not possible in the measurement of a first time and a re-measurement is necessary, and
in the calculating, the blood pressure of the subject is calculated based on a plurality of the pulses determined to be correctly detected in the first determining in the measurement of a first time or a second and subsequent times.

According to the presently disclosed subject matter, even when the blood pressure cannot be calculated due to noise or the like in the plurality of pulses measured in the measurement step of one time, by synthesizing a plurality of pulses measured in the measurement steps of a plurality of times, a possibility that the blood pressure can be calculated can be improved and a burden on the subject can be reduced.

### BRIEF DESCRIPTION OF DRAWINGS

FIG. 1 is a block diagram illustrating a configuration of a physiological information measurement device according to embodiments of the presently disclosed subject matter.

FIG. 2 is a blood pressure measurement processing flow according to a first embodiment of the presently disclosed subject matter.

FIG. 3 is a graph illustrating an example of a pulse detection result of a physiological information measurement device in the related art.

FIG. 4 is a graph illustrating an example of a pulse detection result of a physiological information measurement device according to a first embodiment of the presently disclosed subject matter.

FIG. 5 is a graph when the physiological information measurement device according to the first embodiment of the presently disclosed subject matter ends a measurement step at an early stage.

FIG. 6 is a graph illustrating an example of a pulse detection result when a physiological information measurement device according to a second embodiment of the presently disclosed subject matter ends a measurement step at an early stage.

FIG. 7 is a graph illustrating an example of a pulse detection result obtained using the physiological information measurement device according to the second embodiment of the presently disclosed subject matter when an initial value to be stepwise decreased in a measurement step of a second time is set to be small.

FIG. 8 is a graph when a physiological information measurement device according to a third embodiment of the presently disclosed subject matter ends a measurement step at an early stage.

### DESCRIPTION OF EMBODIMENTS

Hereinafter, embodiments of the presently disclosed subject matter will be described with reference to the drawings. Members having the same reference numerals as those already described in the description of the embodiment will not be described for convenience of description. Further, for convenience of description, dimensions of each member illustrated in the drawings may be different from actual dimensions of each member.

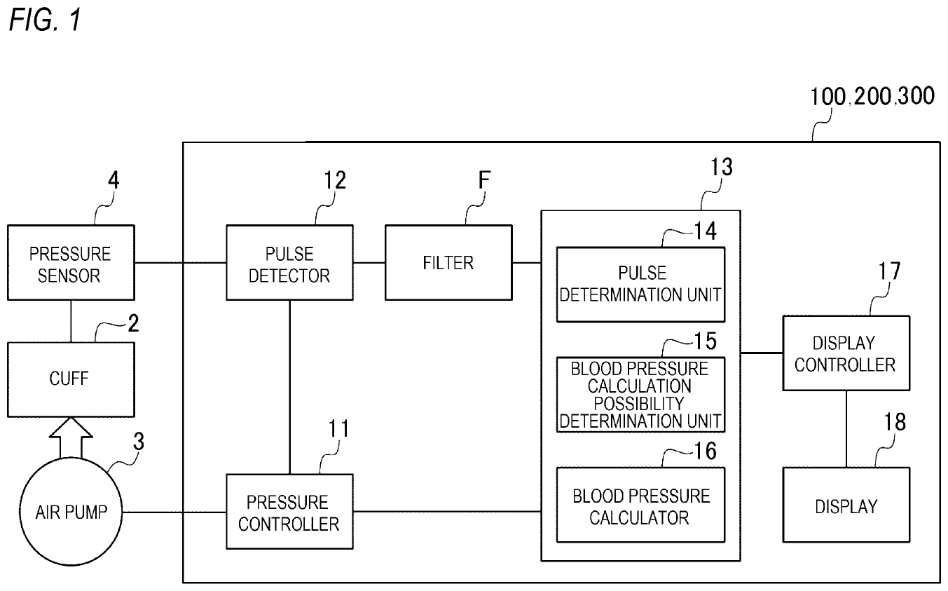

FIG. 1 is a block diagram illustrating a configuration of a physiological information measurement device 100 according to embodiments of the presently disclosed subject matter. The physiological information measurement device 100 can include a pressure controller 11, a pulse detector 12, a filter F, a processor 13, a display controller 17, and a display 18. The processor 13 can include a pulse determination unit 14, a blood pressure calculation possibility determination unit 15, and a blood pressure calculator 16. The physiological information measurement device 100 is configured to be connected to a cuff 2 that is to be wrapped around an upper arm or a lower limb of a subject, an air pump 3 that causes air to flow into the cuff 2, and a pressure sensor 4 that detects pressure vibration generated in the cuff 2. The physiological information measurement device 100 may be configured not to perform filter processing using the filter F. The physiological information measurement device 100 may include the air pump 3 and the pressure sensor 4 therein. Here, the pulse determination unit 14 may be referred as a first determination unit, and the blood pressure calculation possibility determination unit 15 may be referred as a second determination unit.

The pressure controller 11 adjusts an amount of air flowing into the cuff 2 by controlling an applied pressure of the air pump 3. Specifically, the pressure controller 11 performs at least once a measurement step of stepwise decreasing the applied pressure from an initial value (for example, 100 mmHg) (for example, decreasing the applied pressure by 5 mmHg at predetermined intervals). The pressure controller 11 may perform at least once a measurement step of increasing the applied pressure from an initial value (for example, 0 mmHg) at a predetermined rate (for example, increasing the applied pressure by 25 mmHg per second). Further, a method of controlling the applied pressure may be changed for each measurement step, such as stepwise decreasing the pressure in the measurement step of a first time, and increasing the pressure at a predetermined rate in the measurement step of a second time. The pressure controller 11 may control magnitude of the applied pressure or a duration of application of the applied pressure based on the determination result of the pulse determination unit 14.

The pulse detector 12 detects a pulse of the subject (oscillation signal) from the pressure vibration detected by the pressure sensor 4 at a timing when the applied pressure of the cuff 2 is decreased or increased.

The filter F removes noise superimposed on the pulse detected by the pulse detector 12. The filter F may be configured to change a noise removal method according to a level of the superimposed noise.

The pulse determination unit 14 determines whether the pulse from which the noise has been removed by the filter F (the pulse detected by the pulse detector 12 when the physiological information measurement device 100 does not perform the filter processing using the filter F) is a correctly detected pulse, this determination may be referred as first determining. Specifically, it is determined that the pulse is correctly detected when an amplitude or the like of the pulse is within a predetermined range, and it is determined that the pulse is not correctly detected when the amplitude or the like of the pulse is outside the predetermined range. When it is determined that the pulse is not correctly detected, the pulse determination unit 14 discards the pulse.

The blood pressure calculation possibility determination unit 15 determines whether a blood pressure of the subject can be calculated based on a plurality of the pulses determined to be correctly detected by the pulse determination unit 14, this determination may be referred as second determining. Specifically, it is determined whether a systolic period and a diastolic period can be specified from a transition (envelope) of the amplitude of the pulse with respect to the applied pressure of the cuff 2.

When the blood pressure calculation possibility determination unit 15 determines that the blood pressure of the subject can be calculated, the blood pressure calculator 16 specifies the systolic phase and the diastolic phase based on the plurality of pulses determined to be correctly detected by the pulse determination unit 14, and calculates blood pressures (systolic blood pressure (SYS), diastolic blood pressure (DIA), and mean blood pressure (MAP)) of the subject.

The display controller 17 displays, on the display 18, various calculation results (measurement values) and waveforms including the blood pressures calculated by the blood pressure calculator 16. The display 18 is, for example, a liquid crystal display provided in a case of the physiological information measurement device 100. The display 18 may be a touch panel that receives an instruction related to an operation from a user. Further, the display 18 may be configured to be attachable to and detachable from the physiological information measurement device 100, or may be a separate body connected to the physiological information measurement device 100 by a cable, Wi-Fi, or the like.

### First Embodiment

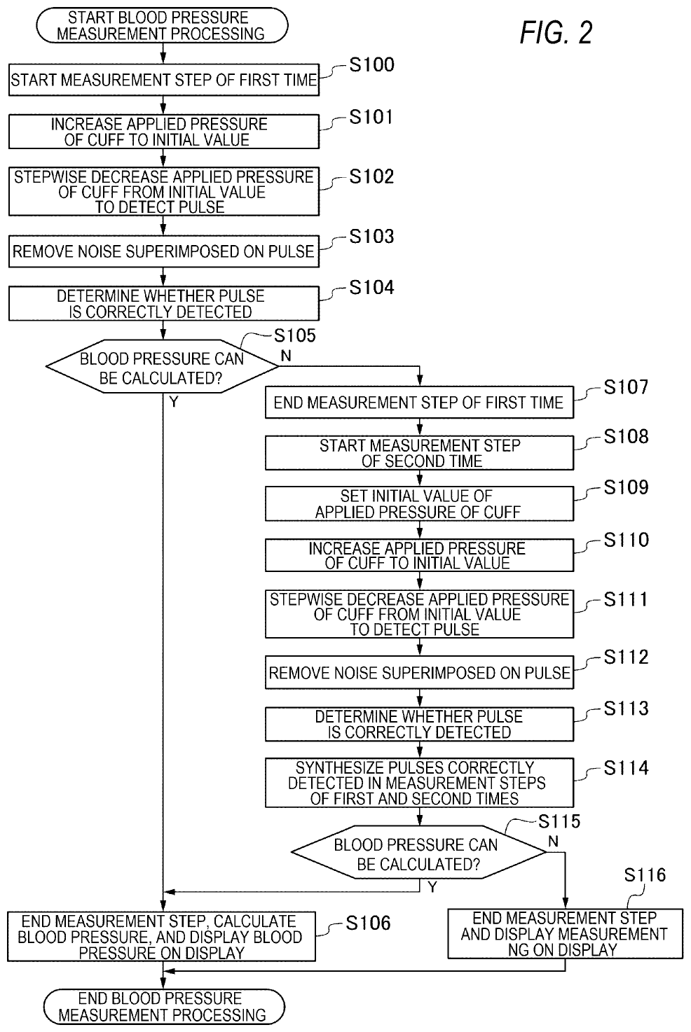

Hereinafter, the physiological information measurement device 100 according to a first embodiment will be described in detail with reference to FIGS. 2 to 5.

FIG. 2 illustrates a blood pressure measurement processing flow according to the physiological information measurement device 100 of the first embodiment of the presently disclosed subject matter. The physiological information measurement device 100 according to the first embodiment is configured to decrease an applied pressure P of the cuff 2 in each measurement step.

When receiving a blood pressure measurement instruction from a user through the display 18 or the like, the physiological information measurement device 100 starts the blood pressure measurement processing and starts the measurement step of a first time (S100).

Next, the pressure controller 11 controls the air pump 3 to increase the applied pressure of the cuff 2 to a preset first initial value (S101). Thereafter, the pressure controller 11 controls the air pump 3 such that the applied pressure of the cuff 2 is stepwise decreased from the first initial value, and the pulse detector 12 detects the pulse of the subject from the pressure vibration detected by the pressure sensor 4 (S102).

Next, the noise superimposed on the pulse detected by the pulse detector 12 is removed by the filter F (S103), and the pulse determination unit 14 determines whether the pulse from which the noise has been removed by the filter F is a correctly detected pulse (S104).

Next, the blood pressure calculation possibility determination unit 15 determines whether the blood pressure of the subject can be calculated based on a plurality of the pulses determined to be correctly detected by the pulse determination unit 14 (S105). When it is determined that the blood pressure of the subject can be calculated (Yes in S105), the physiological information measurement device 100 ends the measurement step and cuts off the applied pressure of the cuff 2, the blood pressure calculator 16 calculates the blood pressure of the subject, and the display controller 17 displays various calculation results including the calculated blood pressure on the display 18 (S106). When it is determined that the blood pressure of the subject cannot be calculated and a re-measurement is necessary (No in S105), the physiological information measurement device 100 ends the measurement step of a first time (S107) and starts the measurement step of a second time (S108). At this time, the blood pressure calculation possibility determination unit 15 holds the plurality of pulses determined to be correctly detected in the measurement step of the first time instead of discarding the pulses.

After the measurement step of the second time is started (S108), the pressure controller 11 sets a second initial value of the applied pressure of the cuff 2 (S109). The second initial value may be the same value as the first initial value, or may be a value newly set based on the determination result of the pulse determination unit 14.

Since subsequent steps S110 to S113 are the same as steps S101 to S104 described above, the description thereof will be omitted.

After it is determined whether the pulse is a correctly detected pulse (S113), the blood pressure calculation possibility determination unit 15 synthesizes the plurality of pulses determined to be correctly detected by the pulse determination unit 14 in the measurement step of the first time and the measurement step of the second time (S114). The blood pressure calculation possibility determination unit 15 determines whether the blood pressure of the subject can be calculated based on the plurality of synthesized pulses (S115). When it is determined that the blood pressure of the subject can be calculated (Yes in S115), the physiological information measurement device 100 ends the measurement step and cuts off the applied pressure of the cuff 2, the blood pressure calculator 16 calculates the blood pressure of the subject, and the display controller 17 displays various calculation results including the calculated blood pressure on the display 18 (S106). When it is determined that the blood pressure of the subject cannot be calculated (No in S115), the physiological information measurement device 100 ends the measurement step, and the display controller 17 displays on the display 18 that the blood pressure cannot be measured and the measurement is ended (S116). Thereafter, the physiological information measurement device 100 ends the blood pressure measurement processing.

In the processing flow illustrated in FIG. 2, when the blood pressure cannot be calculated until the measurement step of the second time, the blood pressure measurement processing is ended by displaying on the display 18 that the blood pressure cannot be measured and the measurement is ended, but the presently disclosed subject matter is not limited thereto. That is, the blood pressure measurement processing may be ended by displaying on the display 18 that the blood pressure cannot be measured and the measurement is ended when the blood pressure cannot be calculated until the measurement step of an Nth time (N is an integer of 3 or more). In this case, steps S107 to S115 are repeated in the measurement steps of third and subsequent times.

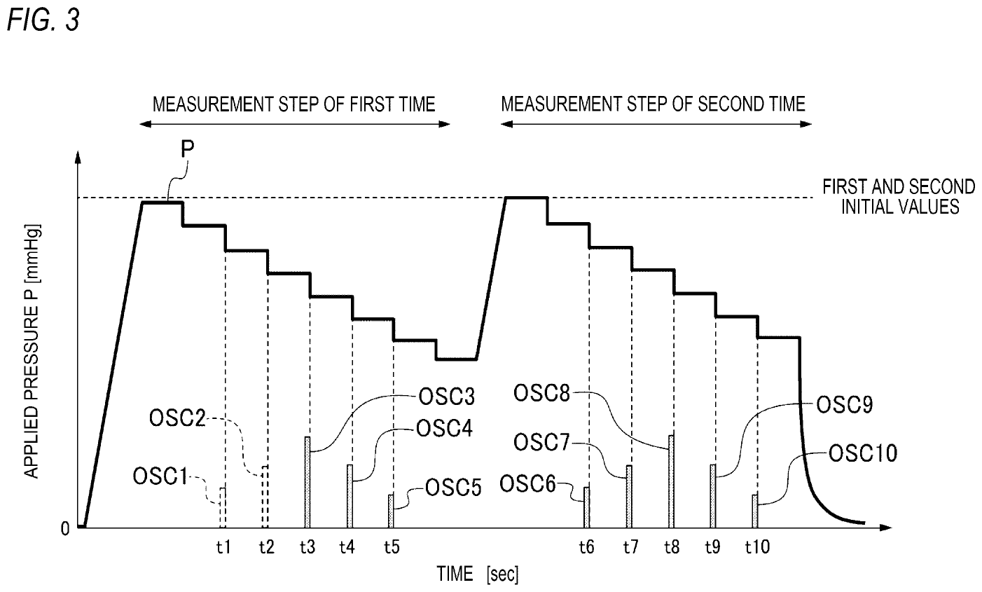

In order to describe a graph illustrating an example of the pulse detection result obtained using the physiological information measurement device 100 according to the first embodiment of the presently disclosed subject matter, for comparison, a graph illustrating an example of a pulse detection result obtained using the physiological information measurement device in the related art will be described with reference to FIG. 3.

In FIG. 3, a horizontal axis represents a time t(s), and a vertical axis represents the applied pressure P (mmHg). In the measurement step of the first time, the applied pressure P of the cuff 2 is stepwise decreased from the first initial value. During this period, the pulse detector 12 detects the pulse of the subject from the pressure vibration detected by the pressure sensor 4. The pulse detector 12 detects pulses OSC1 to OSC5 at time points t1, t2, t3, t4, and t5. In the example illustrated in FIG. 3, the pulses OSC1 and OSC2 are determined not to be correctly detected pulses, and the pulses OSC3 to OSC5 are determined to be correctly detected pulses by the pulse determination unit 14. In the graph illustrated in FIG. 3, since amplitudes of the pulses OSC3 to OSC5 monotonically decrease, the amplitudes of the pulses OSC3 to OSC5 monotonically increase in an envelope, and a peak value of the envelope cannot be specified. Therefore, the systolic period and the diastolic period cannot be specified. Accordingly, the blood pressure calculation possibility determination unit 15 determines that the blood pressure cannot be calculated from the pulses OSC3 to OSC5 and a re-measurement is necessary. At this time, the blood pressure calculation possibility determination unit 15 discards the pulses OSC3 to OSC5 determined to be correctly detected pulses.

In the measurement step of the second time, the applied pressure P of the cuff 2 is stepwise decreased from the second initial value (the same value as the first initial value). In this period, the pulse detector 12 detects pulses OSC6 to OSC10 at time points t6, 17, 18, 19, and t10. In the example illustrated in FIG. 3, the pulses OSC6 to OSC10 are determined to be correctly detected pulses by the pulse determination unit 14. In the graph illustrated in FIG. 3, since amplitudes of the pulses OSC6 to OSC10 have an upwardly convex shape with the amplitude of the pulse OSC8 as an extreme value, the amplitudes of the pulses OSC6 to OSC10 in the envelope have an upwardly convex shape, and the peak value of the envelope can be specified. Therefore, the systolic period and the diastolic period can be specified. Accordingly, the blood pressure calculation possibility determination unit 15 determines that the blood pressure can be calculated from the pulses OSC6 to OSC10, and the blood pressure calculator 16 calculates the blood pressure from the pulses OSC6 to OSC10.

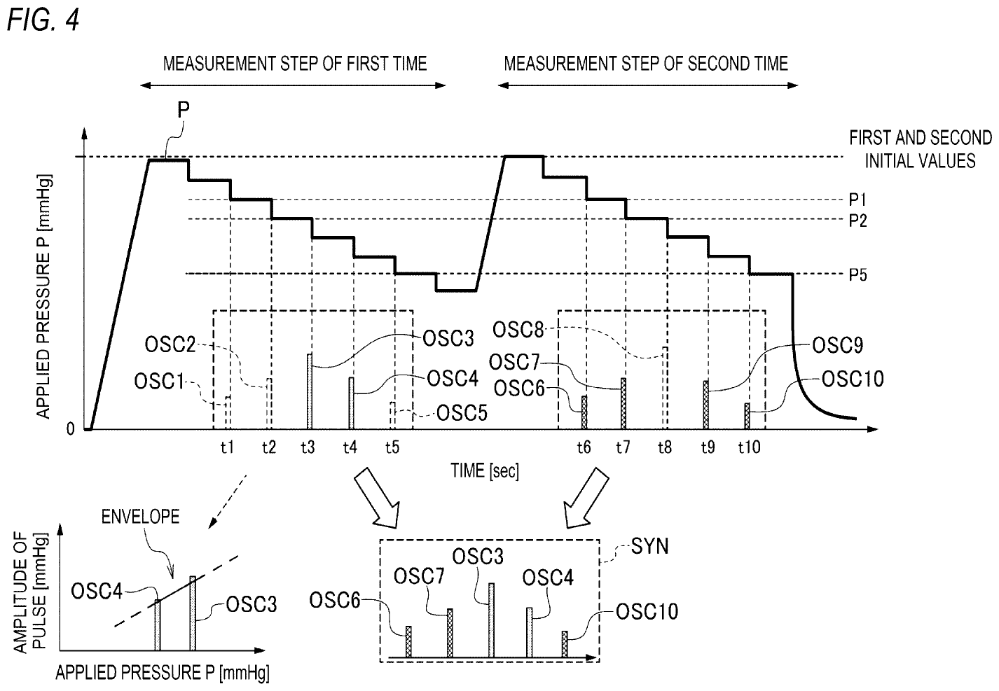

FIG. 4 is a graph illustrating an example of the pulse detection result of the physiological information measurement device 100 according to the first embodiment of the presently disclosed subject matter. A horizontal axis represents a time t(s), and a vertical axis represents the applied pressure P (mmHg).

In the measurement step of the first time, the applied pressure P of the cuff 2 is stepwise decreased from the first initial value. In this period, the pulse detector 12 detects the pulses OSC1 to OSC5 at the time points t1, t2, t3, t4, and t5. In the example illustrated in FIG. 4, the pulses OSC1, OSC2 and OSC5 are determined not to be correctly detected pulses, and the pulses OSC3 and OSC4 are determined to be correctly detected pulses by the pulse determination unit 14. In the graph illustrated in FIG. 4, since amplitudes of the pulses OSC3 and OSC4 monotonically decrease, the amplitudes of the pulses OSC3 and OSC4 monotonically increase in an envelope illustrated in a left lower part of FIG. 4, and a peak value of the envelope cannot be specified. Therefore, the diastolic blood pressure (DIA) and the systolic blood pressure (SYS) cannot be specified.

Accordingly, the blood pressure calculation possibility determination unit 15 determines that the blood pressure cannot be calculated from the pulses OSC3 and OSC4 and a re-measurement is necessary. At this time, the blood pressure calculation possibility determination unit 15 holds the pulses OSC3 and OSC4 determined to be correctly detected pulses.

In the measurement step of the second time, the applied pressure P of the cuff 2 is stepwise decreased from the second initial value (the same value as the first initial value). In this period, the pulse detector 12 detects the pulses OSC6 to OSC10 at time points t6, 17, 18, t9, and t10. In the example illustrated in FIG. 4, the pulse OSC8 is determined not to be a correctly detected pulse, and the pulses OSC6, OSC7, OSC9 and OSC10 are determined to be correctly detected pulses by the pulse determination unit 14.

Next, the blood pressure calculation possibility determination unit 15 synthesizes the pulses OSC3 and OSC4 held in the measurement step of the first time and the pulses OSC6, OSC7, and OSC10 detected in the measurement step of the second time, as illustrated in a lower center part of FIG. 4. Here, the applied pressures P of the cuff 2 at the pulses OSC1, OSC2, and OSC5 determined not to be correctly detected in the measurement step of the first time are set as P1, P2, and P5. In the measurement step of the second time, the pulses OSC6, OSC7, and OSC10 which are detected at the same level of applied pressure as P1, P2, and P5 and determined to be correctly detected are used as data for synthesis. In the graph illustrated in the lower center part of FIG. 4, since the synthesized pulses OSC6, OSC7, OSC3, OSC4, and OSC10 (hereinafter also referred to as synthesized data SYN) have an upwardly convex shape with the amplitude of the pulse OSC3 as an extreme value, the amplitudes of the pulses OSC6, OSC7, OSC3, OSC4, and OSC10 also have an upwardly convex shape in the envelope, and the peak value of the envelope can be specified. Therefore, the diastolic blood pressure (DIA) and the systolic blood pressure (SYS) can be specified. Accordingly, the blood pressure calculation possibility determination unit 15 determines that the blood pressure can be calculated from the synthesized data SYN, and the blood pressure calculator 16 calculates the blood pressure from the synthesized data SYN.

As described above, even when the blood pressure cannot be calculated due to noise or the like in the plurality of pulses measured in the measurement step of one time, by synthesizing a plurality of pulses measured in the measurement steps of a plurality of times, the physiological information measurement device 100 according to the present embodiment can improve a possibility that the blood pressure can be calculated and reduce a burden on the subject.

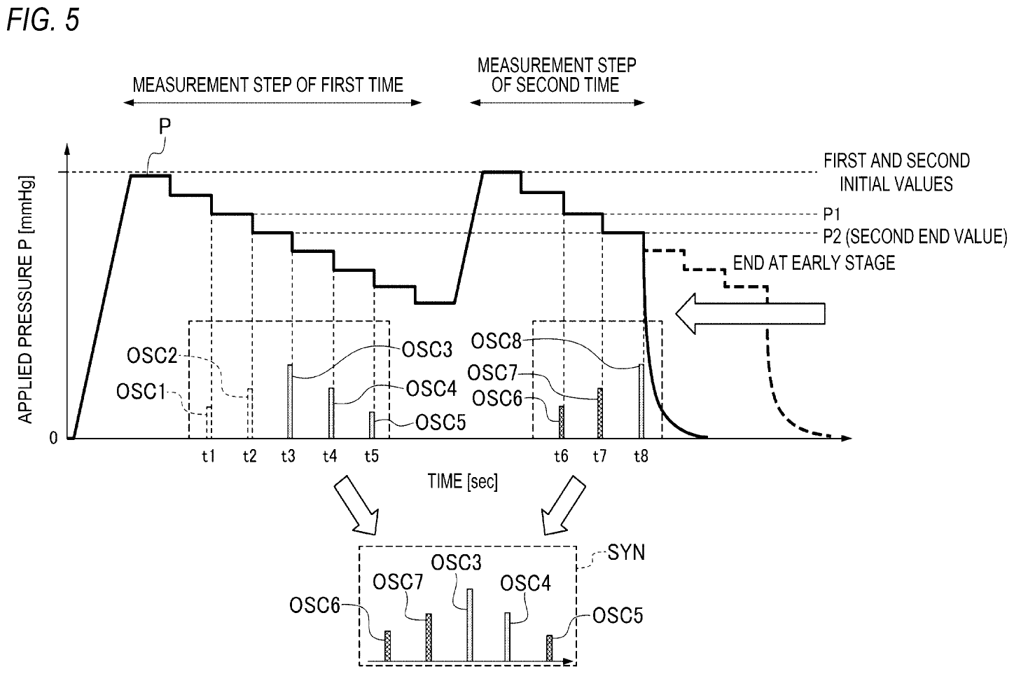

FIG. 5 is a graph when the physiological information measurement device 100 according to the first embodiment of the presently disclosed subject matter ends the measurement step at an early stage. A horizontal axis represents a time t(s), and a vertical axis represents the applied pressure P (mmHg).

In the graph illustrated in FIG. 5, in the measurement step of the first time, it is determined that the pulses OSC3 to OSC5 among the detected pulses OSC1 to OSC5 are correctly detected pulses. In the measurement step of the second time, it is determined that all of the detected pulses OSC6 to OSC8 are correctly detected pulses.

The blood pressure calculation possibility determination unit 15 synthesizes the pulses OSC3 to OSC5 held in the measurement step of the first time and the pulses OSC6 and OSC7 detected in the measurement step of the second time to create the synthesized data SYN, as illustrated in a lower part of FIG. 5. Here, the applied pressures P of the cuff 2 at the pulses OSC1 and OSC2 determined not to be correctly detected in the measurement step of the first time are set as P1 and P2. In the measurement step of the second time, the pulses OSC6 and OSC7 which are detected at the same level of applied pressure as P1 and P2 and determined to be correctly detected are used as data for synthesis. As in the case illustrated in FIG. 4, the blood pressure calculation possibility determination unit 15 determines that the blood pressure can be calculated from the synthesized data SYN, and the blood pressure calculator 16 calculates the blood pressure from the synthesized data SYN.

Here, the time point t8 at which the pulse OSC8 is detected is in the middle of executing a pressure decreasing step in the measurement step of the second time. However, since the diastolic blood pressure (DIA) can be specified in the measurement step of the first time, and the blood pressure calculator 16 can calculate the blood pressure from the synthesized data SYN, the pressure controller 11 cuts off the applied pressure P at a second end value (applied pressure P2), and ends the measurement step of the second time at an early stage, as illustrated in FIG. 5.

As described above, when the pulse necessary for calculating the blood pressure is correctly detected and it is determined that the blood pressure can be calculated, the physiological information measurement device 100 according to the present embodiment can shorten a measurement time and reduce a burden on the subject by ending the measurement step at an early stage. As an example of controlling a duration of application of the applied pressure may include to short a measurement time.

### Second Embodiment

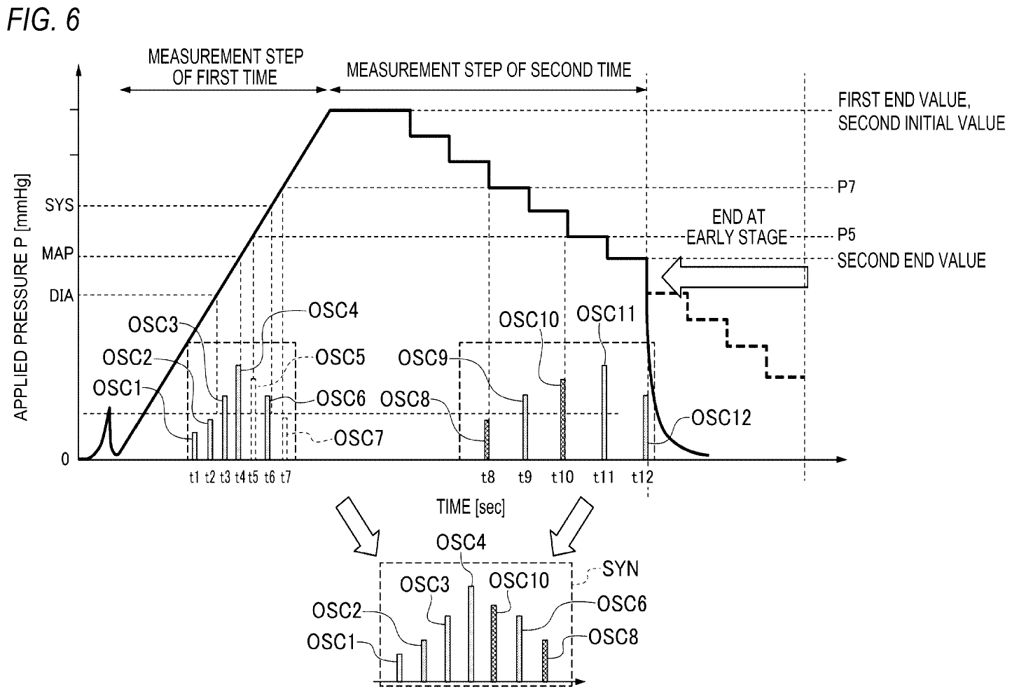

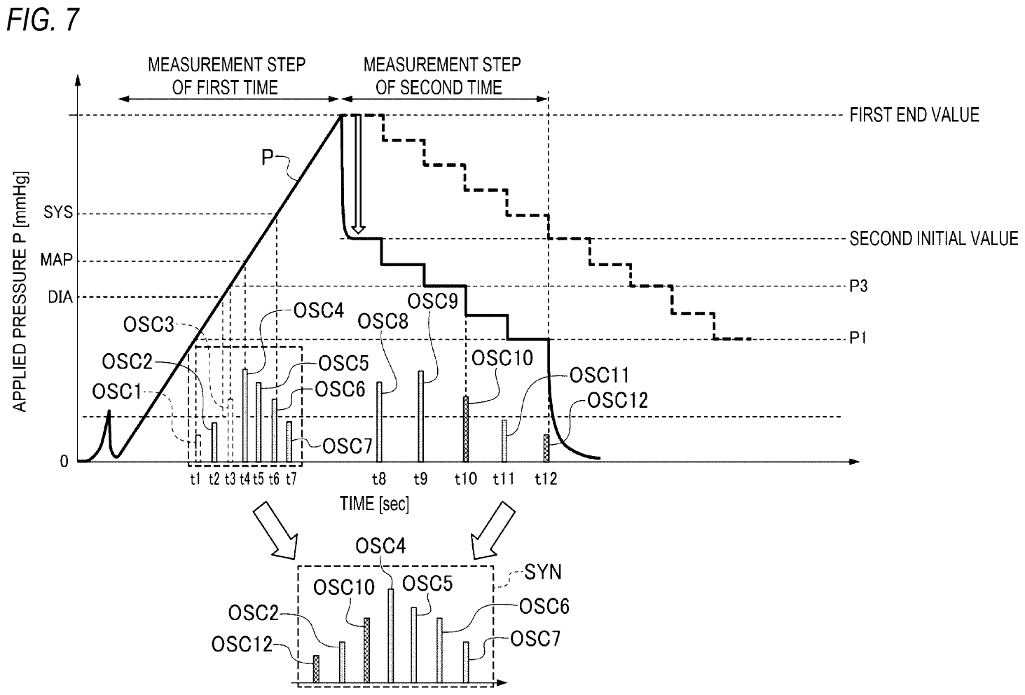

The physiological information measurement device 100 according to the first embodiment described above calculates the blood pressure by stepwise decreasing the applied pressure P of the cuff 2 in the measurement step of the first time and the measurement step of the second time. Meanwhile, in a physiological information measurement device according to a second embodiment, the blood pressure is calculated by increasing the applied pressure P of the cuff 2 at a predetermined rate in the measurement step of a first time, and stepwise decreasing the applied pressure P of the cuff 2 in the measurement step of a second time. Hereinafter, a physiological information measurement device 200 according to the second embodiment will be described in detail with reference to FIGS. 6 and 7.

FIG. 6 is a graph illustrating an example of a pulse detection result when the physiological information measurement device 200 according to the second embodiment of the presently disclosed subject matter ends the measurement step at an early stage. A horizontal axis represents a time t(s), and a vertical axis represents the applied pressure P (mmHg). In the measurement step of the first time, the applied pressure P of the cuff 2 is increased from a first initial value to a first end value at a predetermined rate. In this period, the pulse detector 12 detects the pulses OSC1 to OSC7 at the time points t1, t2, t3, t4, t5, t6, and t7. In the example illustrated in FIG. 6, the pulses OSC5 and OSC7 are determined not to be correctly detected pulses, and the pulses OSC1 to OSC4 and OSC6 are determined to be correctly detected pulses by the pulse determination unit 14. From amplitudes of the pulses OSC1 to OSC4, and OSC6, the mean blood pressure (MAP) and the diastolic blood pressure (DIA) can be specified, but the systolic blood pressure (SYS) cannot be specified.

Accordingly, the blood pressure calculation possibility determination unit 15 determines that the blood pressure cannot be calculated from the pulses OSC1 to OSC4, and OSC6 and a re-measurement is necessary. At this time, the blood pressure calculation possibility determination unit 15 holds the pulses OSC1 to OSC4, and OSC6 determined to be correctly detected pulses.

In the measurement step of the second time, the applied pressure P of the cuff 2 is stepwise decreased from a second initial value (the same value as the first end value). In this period, the pulse detector 12 detects pulses OSC8 to OSC12 at time points t8, 19, t10, t11, and t12. In the example illustrated in FIG. 6, the pulses OSC8 to OSC12 are determined to be correctly detected pulses by the pulse determination unit 14.

Next, the blood pressure calculation possibility determination unit 15 synthesizes the pulses OSC1 to OSC4 and OSC6 held in the measurement step of the first time and the pulses OSC8 and OSC10 detected in the measurement step of the second time, as illustrated in a lower part of FIG. 6. Here, the applied pressures P of the cuff 2 at the pulses OSC5 and OSC7 determined not to be correctly detected in the measurement step of the first time are set as P5 and P7. In the measurement step of the second time, the pulses OSC10 and OSC8 which are detected at the same level of applied pressure as P5 and P7 and determined to be correctly detected are used as data for synthesis. The systolic blood pressure (SYS) and the diastolic blood pressure (DIA) can be specified from the synthesized data SYN. Accordingly, the blood pressure calculation possibility determination unit 15 determines that the blood pressure can be calculated from the synthesized data SYN, and the blood pressure calculator 16 calculates the blood pressure from the synthesized data SYN.

Here, the time point t12 at which the pulse OSC12 is detected is in the middle of executing a pressure decreasing step in the measurement step of the second time. However, since the blood pressure calculator 16 can calculate the blood pressure from the synthesized data SYN, the pressure controller 11 cuts off the applied pressure P at a second end value, and ends the measurement step of the second time at an early stage, as illustrated in FIG. 6.

As described above, when the pulse necessary for calculating the blood pressure is correctly detected and it is determined that the blood pressure can be calculated, the physiological information measurement device 200 according to the present embodiment can shorten a measurement time and reduce a burden on the subject by ending the measurement step at an early stage.

FIG. 7 is a graph illustrating an example of a pulse detection result obtained using the physiological information measurement device 200 according to the second embodiment of the presently disclosed subject matter when an initial value to be stepwise decreased in the measurement step of the second time is set to be small. A horizontal axis represents a time t(s), and a vertical axis represents the applied pressure P (mmHg).

In the measurement step of the first time, the applied pressure P of the cuff 2 is increased from a first initial value to a first end value at a predetermined rate. In this period, the pulse detector 12 detects the pulses OSC1 to OSC7 at the time points t1, t2, t3, t4, t5, t6, and t7. In the example illustrated in FIG. 7, the pulses OSC1 and OSC3 are determined not to be correctly detected pulses, and the pulses OSC2, OSC4 to OSC7 are determined to be correctly detected pulses by the pulse determination unit 14. From amplitudes of the pulses OSC2, and OSC4 to OSC7, the mean blood pressure (MAP) and the systolic blood pressure (SYS) can be specified, but the diastolic blood pressure (DIA) cannot be specified.

Accordingly, the blood pressure calculation possibility determination unit 15 determines that the blood pressure cannot be calculated from the pulses OSC2 and OSC4 to OSC7 and a re-measurement is necessary. At this time, the blood pressure calculation possibility determination unit 15 holds the pulses OSC2 and OSC4 to OSC7 determined to be correctly detected pulses.

In the measurement step of a second time, the pressure controller 11 sets a second initial value of the applied pressure P in the measurement step based on the determination result of the blood pressure calculation possibility determination unit 15 in the measurement step of the first time. At this time, the pressure controller 11 may set, as the second initial value, a minimum value within a range in which the pulse (pulse that cannot be measured in the measurement step of the first time) necessary for calculating the blood pressure can be detected. In the example of FIG. 7, a value larger than the mean blood pressure (MAP) specified in the measurement step of the first time by a predetermined value (for example, 10 mmHg) is set as the second initial value. The applied pressure P of the cuff 2 is decreased stepwise from the second initial value. In this period, the pulse detector 12 detects the pulses OSC8 to OSC12 at the time points t8, 19, t10, t11, and t12. In the example illustrated in FIG. 7, the pulses OSC8 to OSC12 are determined to be correctly detected pulses by the pulse determination unit 14.

Next, the blood pressure calculation possibility determination unit 15 synthesizes the pulses OSC2, and OSC4 to OSC7 held in the measurement step of the first time and the pulses OSC10 and OSC12 detected in the measurement step of the second time, as illustrated in a lower part of FIG. 7. Here, the applied pressures P of the cuff 2 at the pulses OSC1 and OSC3 determined not to be correctly detected in the measurement step of the first time are set as P1 and P3. In the measurement step of the second time, the pulses OSC12 and OSC10 which are detected at the same level of applied pressure as P1 and P3 and determined to be correctly detected are used as data for synthesis. The systolic blood pressure (SYS) and the diastolic blood pressure (DIA) can be specified from the synthesized data SYN. Accordingly, the blood pressure calculation possibility determination unit 15 determines that the blood pressure can be calculated from the synthesized data SYN, and the blood pressure calculator 16 calculates the blood pressure from the synthesized data SYN.

As described above, when a part of a plurality of pulses necessary for calculating the blood pressure is not correctly detected, the physiological information measurement device 200 according to the present embodiment resets the second initial value of the applied pressure in the measurement step of a next time based on the determination result to shorten the measurement time, and thus a burden on the subject can be reduced.

### Third Embodiment

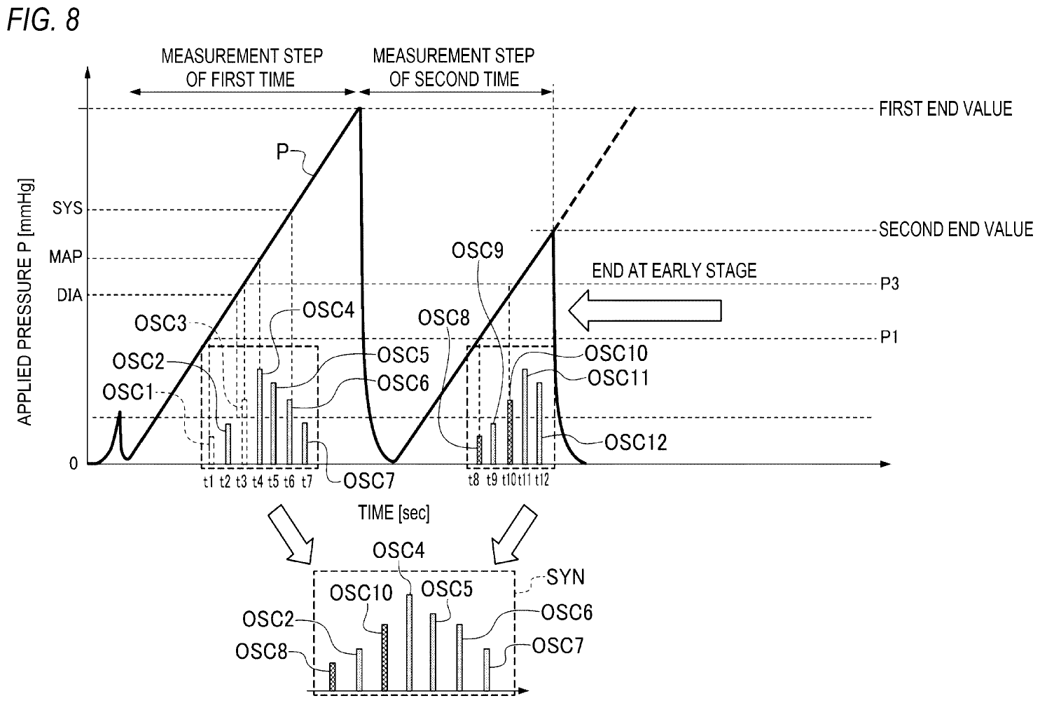

The physiological information measurement device 100 according to the first embodiment described above calculates the blood pressure by stepwise decreasing the applied pressure P of the cuff 2 in the measurement step of the first time and the measurement step of the second time. Meanwhile, in a physiological information measurement device according to a third embodiment, the blood pressure is calculated by increasing the applied pressure P of the cuff 2 at a predetermined rate in the measurement step of a first time and the measurement step of a second time. Hereinafter, a physiological information measurement device 300 according to the third embodiment will be described in detail with reference to FIG. 8.

FIG. 8 is a graph when the physiological information measurement device according to the third embodiment of the presently disclosed subject matter ends the measurement step at an early stage. A horizontal axis represents a time t(s), and a vertical axis represents the applied pressure P (mmHg).

In the measurement step of the first time, the applied pressure P of the cuff 2 is increased from a first initial value to a first end value at a predetermined rate. In this period, the pulse detector 12 detects the pulses OSC1 to OSC7 at the time points t1, t2, t3, t4, t5, t6, and t7. In the example illustrated in FIG. 8, the pulses OSC1 and OSC3 are determined not to be correctly detected pulses, and the pulses OSC2, OSC4 to OSC7 are determined to be correctly detected pulses by the pulse determination unit 14. From amplitudes of the pulses OSC2, and OSC4 to OSC7, the mean blood pressure (MAP) and the systolic blood pressure (SYS) can be specified, but the diastolic blood pressure (DIA) cannot be specified.

Accordingly, the blood pressure calculation possibility determination unit 15 determines that the blood pressure cannot be calculated from the pulses OSC2 and OSC4 to OSC7 and a re-measurement is necessary. At this time, the blood pressure calculation possibility determination unit 15 holds the pulses OSC2 and OSC4 to OSC7 determined to be correctly detected pulses.

In the measurement step of the second time, the applied pressure P of the cuff 2 is increased from a second initial value to a second end value at a predetermined rate. In this period, the pulse detector 12 detects the pulses OSC8 to OSC12 at the time points t8, 19, t10, t11, and t12. In the example illustrated in FIG. 8, the pulses OSC8 to OSC12 are determined to be correctly detected pulses by the pulse determination unit 14.

Next, the blood pressure calculation possibility determination unit 15 synthesizes the pulses OSC2 and OSC4 to OSC7 held in the measurement step of the first time and the pulses OSC8 and OSC10 detected in the measurement step of the second time, as illustrated in a lower part of FIG. 8. Here, the applied pressures P of the cuff 2 at the pulses OSC1 and OSC3 determined not to be correctly detected in the measurement step of the first time are set as P1 and P3. In the measurement step of the second time, the pulses OSC8 and OSC10 which are detected at the same level of applied pressure as P1 and P3 and determined to be correctly detected are used as data for synthesis. The systolic blood pressure (SYS) and the diastolic blood pressure (DIA) can be specified from the synthesized data SYN. Accordingly, the blood pressure calculation possibility determination unit 15 determines that the blood pressure can be calculated from the synthesized data SYN, and the blood pressure calculator 16 calculates the blood pressure from the synthesized data SYN.

Here, the time point t12 at which the pulse OSC12 is detected is in the middle of executing a pressure increasing step in the measurement step of the second time. However, since the blood pressure calculator 16 can calculate the blood pressure from the synthesized data SYN, the pressure controller 11 cuts off the applied pressure P at the second end value, and ends the measurement step of the second time at an early stage, as illustrated in FIG. 8.

As described above, when the pulse necessary for calculating the blood pressure is correctly detected and it is determined that the blood pressure can be calculated, the physiological information measurement device 300 according to the present embodiment can shorten a measurement time and reduce a burden on the subject by ending the measurement step at an early stage.

Although the embodiments of the presently disclosed subject matter have been described above, it is needless to say that the technical scope of the presently disclosed subject matter should not be construed as being limited to the description of the embodiments. The present embodiments are merely examples, and it is understood by those skilled in the art that various modifications of the embodiments are possible within the scope of the disclosed subject matters described in the claims. The technical scope of the presently disclosed subject matter should be determined based on the scope of the disclosed subject matters described in the claims and equivalents thereof.

For example, in the physiological information measurement devices 100, 200, and 300 according to the embodiments of the presently disclosed subject matter, when the blood pressure calculation possibility determination unit 15 determines that the blood pressure cannot be calculated in the middle of the measurement step of a certain time and a re-measurement is necessary, the pressure controller 11 may end the measurement step and start the measurement step of a next time at the time when it is determined that the blood pressure cannot be calculated.

Further, the processes of the physiological information measurement devices 100, 200, and 300 according to the embodiments can be implemented as a computer program that operates in the physiological information measurement devices 100, 200, and 300. That is, the physiological information measurement devices 100, 200, and 300 each can include a processor such as a CPU and a memory.

The program is stored in a non-transitory computer-readable medium and can be read by a computer. Examples of the non-transitory computer-readable medium include a magnetic recording medium, a magneto-optical recording medium, a CD-ROM, a CD-R, a CD-R/W, and a semi-conductor memory (including an EPROM and a flash ROM). Further, the program may be read by a computer using various types of temporary computer-readable media. Examples of the temporary computer-readable medium include an electric signal, an optical signal, and an electromagnetic wave. The temporary computer-readable medium can supply a program to the computer via a wired communication path such as an electric wire and an optical fiber or a wireless communication path.

1. A physiological information measurement device comprising:
a pressure controller configured to execute a measurement of increasing or decreasing an applied pressure of a cuff with respect to a predetermined site of a subject from an initial value;
a pulse detector configured to detect a pulse from a pressure received by the cuff from the predetermined site;
a pulse determination unit configured to determine whether the pulse detected by the pulse detector is a correctly detected pulse;
a blood pressure calculation possibility determination unit configured to determine whether calculation of a blood pressure of the subject is possible based on the pulse detected by the pulse detector; and
a blood pressure calculator configured to calculate the blood pressure of the subject based on a plurality of the pulses determined to be correctly detected by the pulse determination unit, wherein
the pressure controller executes at least the measurement of a second time when the blood pressure calculation possibility determination unit determines that the calculation of the blood pressure is not possible in the measurement of a first time and a re-measurement is necessary, and
the blood pressure calculator calculates the blood pressure of the subject based on a plurality of the pulses determined to be correctly detected by the pulse determination unit in the measurement of a first time or a second and subsequent times.

2. The physiological information measurement device according to claim 1, wherein
when the blood pressure calculation possibility determination unit determines that the calculation of the blood pressure is possible, the pressure controller cuts off the applied pressure and ends the measurement.

3. The physiological information measurement device according to claim 1, wherein
when the blood pressure calculation possibility determination unit determines that the calculation of the blood pressure is not possible in the measurement of a certain time and a re-measurement is necessary, the pressure controller ends the measurement and starts the measurement of a next time.

4. The physiological information measurement device according to claim 1, wherein
when the blood pressure calculation possibility determination unit determines that the calculation of the blood pressure is not possible in the measurement of a certain time and a re-measurement is necessary, the pressure controller sets the initial value of the applied pressure in the measurement of a next time based on the determination result of the blood pressure calculation possibility determination unit.

5. The physiological information measurement device according to claim 4, wherein
when the blood pressure calculation possibility determination unit determines that the calculation of the blood pressure is not possible in the measurement of a certain time and a re-measurement is necessary, the pressure controller sets a minimum value within a range, in which the pulse necessary for calculating the blood pressure is detectable, as the initial value of the applied pressure in the measurement of the next time.

6. The physiological information measurement device according to claim 1, further comprising:
a filter, wherein
the pulse determination unit determines whether the pulse filtered by the filter is a correctly detected pulse.

7. A physiological information measurement device comprising:
a pressure controller configured to control an applied pressure of a cuff with respect to a predetermined site of a subject;
a pulse detector configured to detect a pulse from a pressure received by the cuff from the predetermined site;
a pulse determination unit configured to determine whether the pulse detected by the pulse detector is a correctly detected pulse; and
a blood pressure calculator configured to calculate a blood pressure of the subject based on a plurality of the pulses determined to be correctly detected by the pulse determination unit, wherein
the pressure controller controls magnitude of the applied pressure or a duration of application of the applied pressure based on the determination result of the pulse determination unit.

8. A physiological information measurement method comprising:
executing a measurement of increasing or decreasing an applied pressure of a cuff with respect to a predetermined site of a subject from an initial value;
detecting a pulse from a pressure received by the cuff from the predetermined site;
first determining whether the pulse detected in the detecting is a correctly detected pulse;
second determining whether calculation of a blood pressure of the subject is possible based on the pulse detected in the detecting; and
calculating the blood pressure of the subject based on a plurality of the pulses determined to be correctly detected in the first determining, wherein
in the executing, at least the measurement of a second time is executed when it is determined in the second determining that the calculation of the blood pressure is not possible in the measurement of a first time and a re-measurement is necessary, and
in the calculating, the blood pressure of the subject is calculated based on a plurality of the pulses determined to be correctly detected in the first determining in the measurement of a first time or a second and subsequent times.

9. A non-transitory computer readable storage medium storing a program for causing a computer to execute a physiological information measurement method according to claim 8.
